(19) United States
(12) Patent Application Publication
(10) Pub. No.: US 2026/0301902 A1
(43) Pub. Date: Oct. 1, 2026

(54) SYSTEM AND METHOD FOR STRUCTURED NARCOTIC CESSATION USING INGESTION MODALITY REDUCTION AND DOSAGE TAPERING FRAMEWORK

(71) Applicant: James William Shaughnessy, Port Washington, NY (US)

(72) Inventor: James William Shaughnessy, Port Washington, NY (US)

(21) Appl. No.: 19/096,725

(22) Filed: Apr. 1, 2025

(51) Int. Cl. G16H 20/10; A61B 5/00; G16H 10/60; G16H 70/20; G16H 80/00

(52) U.S. Cl. CPC G16H 20/10; A61B 5/4845; G16H 10/60; G16H 70/20; G16H 80/00

(57) ABSTRACT

The invention provides a structured framework and associated technology system for narcotic cessation through controlled reduction in ingestion method intensity and dosage tapering. Specifically, it defines a therapeutic protocol involving transitioning patients down an ordered ingestion-intensity ladder—from highly reinforcing methods such as injection, through intermediate modalities such as insufflation and oral ingestion, to minimally reinforcing methods like transdermal patches—while concurrently reducing drug dosage. Implemented through an integrated technological platform comprising software and optional hardware components, the system dynamically guides patients, tracks adherence, provides clinician oversight, and integrates behavioral reinforcement strategies such as randomized rewards and gamified progress tracking. This dual-axis approach to narcotic cessation significantly addresses the limitations of existing treatment methodologies by personalizing intervention, minimizing withdrawal symptoms, and enhancing patient compliance and engagement. The invention is particularly applicable in clinical treatment programs addressing opioid dependency and other substance use disorders.

### TECHNICAL FIELD

The present invention relates to the field of behavioral health and substance use disorder treatment. More particularly, it pertains to systems and methods for structured narcotic cessation, involving phased reduction in substance ingestion intensity and dosage, optionally implemented via software, devices, or clinical support systems.

### BACKGROUND OF THE INVENTION

Substance use disorder (SUD), particularly involving narcotics such as opioids, remains a major global health crisis with profound socioeconomic and public health implications. Traditional treatment methods for narcotic dependence have evolved over the decades, ranging from complete abstinence models to harm-reduction strategies such as medication-assisted therapy (MAT), including the use of methadone and buprenorphine. Despite these advancements, a substantial number of individuals struggle to achieve or maintain long-term cessation due to limitations in current treatment modalities, behavioral relapse, or psychological dependence.

Existing cessation strategies generally fall into one of several categories. The first is the abstinence-based model, which promotes immediate and complete discontinuation of drug use. This approach, while effective for some, often results in severe withdrawal symptoms and high relapse rates, especially in the absence of supportive psychological and medical care. The second is substitution therapy, wherein a medically supervised administration of a longer-acting but less euphoric narcotic (e.g., methadone, buprenorphine, or suboxone) is used to stabilize the patient before attempting tapering. This approach addresses physical dependence but often leads to prolonged use of the substitute substance and does not offer a structured path toward total cessation.

Behavioral therapies, counseling, and support systems are also widely employed. These are critical components of a holistic treatment plan but are often implemented in a generalized or nonspecific manner. As a result, patients may lack a tangible, actionable roadmap that addresses not only what substance is used, but how it is consumed—a critical factor in the reinforcement and psychological reward cycle of addiction.

One aspect that is often underappreciated in current methods is the role of ingestion modality—the way in which a substance is introduced into the body. Different ingestion methods offer varying speeds of onset, peak intensity, and bioavailability. For example, intravenous injection results in nearly instantaneous and highly potent effects, whereas transdermal patches deliver a steady and slow release. These differences significantly influence the addictive potential of a substance. Despite this, most treatment regimens focus solely on reducing dose or switching substances, with little attention paid to the ingestion route itself as a manipulable factor in treatment.

Further complicating the problem is the variability in patient behavior. Individuals may start with one ingestion method and escalate to more intense forms as tolerance builds. Others may oscillate between methods based on access, context, or psychological triggers. A rigid, one-size-fits-all cessation model fails to accommodate this behavioral complexity. Consequently, relapse becomes more likely when patients feel that treatment lacks personalization, is too abrupt, or fails to address their psychological attachment to specific rituals associated with drug use.

Moreover, there is limited use of structured, repeatable frameworks that allow for a gradual shift not only in dose but in the behavioral pattern of drug ingestion. There is also a paucity of technological systems—whether digital, physical, or hybrid—that can systematically guide a patient through a pre-defined ladder of ingestion modalities, each representing a step-down in addiction reinforcement potential. Current mobile health apps may offer dosage reminders or general counseling support, but they do not offer dynamic, structured ingestion-modality tapering frameworks.

The healthcare industry also lacks widely adopted tools for quantifying and standardizing the relative intensity of different ingestion methods. The absence of such a standard framework impedes the development of reproducible, scalable intervention programs that address the behavioral and physiological components of addiction in tandem.

From a clinical perspective, there is a pressing need for interventions that are both structured and flexible—allowing clinicians to guide patients down a path of decreased risk without demanding immediate abstinence. Behavioral and physiological adaptations to drug use vary widely among individuals, requiring a system that can accommodate gradual, controlled change across multiple vectors of addiction: substance type, dose, and ingestion method.

Finally, relapse prevention is significantly hindered by boredom, lack of engagement, and the perceived rigidity of treatment protocols. There exists an unmet need for systems that incorporate behavioral science—such as randomized reinforcement, gamification, or user agency—to improve patient compliance and reduce dropout rates.

In summary, while narcotic cessation has been approached from multiple therapeutic angles, existing methods fall short in offering a unified, stepwise protocol that directly addresses ingestion method intensity, dose reduction, and patient behavior. There is no current system that combines these factors into a singular, actionable framework supported by technological tools for real-time implementation and monitoring.

### SUMMARY OF THE INVENTION

In light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole.

The present invention provides a novel, structured system and method for narcotic cessation that leverages a dual-axis tapering approach: (1) progressive reduction in the intensity of ingestion modality, and (2) gradual tapering of the administered dose. The invention introduces an ordered “ingestion ladder” which ranks methods of drug consumption based on their relative intensity, speed of onset, and addiction reinforcement potential. Patients transition down this ladder in a stepwise manner, switching from more intense methods (e.g., injection) to milder methods (e.g., transdermal patch), while simultaneously reducing dosage. This framework provides a psychologically and physiologically manageable path to cessation that better aligns with patient behavior and neuroadaptation patterns.

In one embodiment, the method is implemented via a digital platform or physical system that monitors patient adherence, suggests transitions, and optionally incorporates behavioral science mechanisms such as random reinforcement or gamification to increase engagement. The system can further integrate a smart drug dispenser or therapeutic support app that dynamically adjusts dosage and ingestion recommendations based on patient input and clinician oversight. This invention provides clinicians and patients with a unified, adaptive roadmap for narcotic cessation, significantly improving treatment adherence, personalizing care, and reducing relapse rates.

This summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following detailed description and figures.

The abovementioned embodiments and further variations of the proposed invention are discussed further in the detailed description.

### BRIEF DESCRIPTION OF THE DRAWINGS

The accompanying drawings, which are incorporated herein and constitute a part of the specification, illustrate embodiments of the present invention and, together with the description, serve to explain the principles of the invention.

FIG. 1 illustrates an ingestion modality hierarchy (also referred to as the “ingestion ladder”) 100 used in the system and method of narcotic cessation according to the embodiments of the present disclosure.

FIG. 2 is a flowchart 200 representing the high-level therapeutic process followed in the Shaughnessy Method of Narcotic Cessation according to the embodiments of the present disclosure.

FIG. 3 presents an expanded procedural flow 300 of the method for narcotic cessation according to the embodiments of the present disclosure.

FIG. 4 is a block diagram 400 of the narcotic cessation system, showing the core components of the computing device used to implement the method according to the embodiments of the present disclosure.

The drawings described herein are for illustration purposes only and are not intended to limit the scope of the present subject matter in any way according to the embodiments of the present disclosure.

### DETAILED DESCRIPTION

In the following description of the embodiments of the invention, reference is made to the accompanying drawings that form a part hereof, and in which are shown by way of illustration specific embodiments in which the invention may be practiced. These embodiments are described in sufficient detail to enable those skilled in the art to practice the invention, and it is to be understood that other embodiments maybe utilized and that changes may be made without departing from the scope of the present invention. The following detailed description is, therefore, not to be taken in a limited sense, and the scope of the present invention is defined only by the appended claims.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the foregoing sections, some features are grouped together in a single embodiment for streamlining the disclosure. This method of disclosure is not to be interpreted as reflecting an intention that the disclosed embodiments of the present disclosure must use more features than are expressly recited in each claim. Rather, as the following claims reflect, the inventive subject matter lies in less than all features of a single disclosed embodiment. Thus, the following claims are hereby incorporated into the detailed description, with each claim standing on its own as a separate embodiment.

Current therapeutic methodologies for narcotic cessation, particularly those dealing with opioids and similar substances, face significant limitations regarding treatment efficacy, patient compliance, relapse prevention, and long-term success. Traditional methods, including abstinence-only models or basic medication-assisted therapies, often lead to high dropout rates, severe withdrawal symptoms, psychological resistance, and relapse due to abrupt cessation or inadequate management of patient-specific behavioral patterns. The absence of structured, stepwise protocols targeting both dosage and ingestion methodology concurrently exacerbates these issues. Moreover, there is a substantial gap in clinically validated, technology-integrated treatment systems that actively guide and monitor patients throughout the cessation process in an individualized and adaptable manner.

The present invention provides a structured framework and technology-based system designed for narcotic cessation through controlled reduction in ingestion method intensity and systematic dosage tapering. It specifically defines a therapeutic protocol whereby patients transition down an ordered ingestion-intensity hierarchy—from highly reinforcing methods such as injection, to intermediate methods like insufflation and oral ingestion, and finally to minimally reinforcing methods such as transdermal patches—while concurrently reducing narcotic dosage. This method can be effectively implemented via an integrated technological platform comprising software, optional hardware components, and clinician oversight. The system dynamically guides patients, tracks adherence, provides clinician monitoring capabilities, and incorporates behavioral reinforcement strategies, including randomized rewards and gamified progress tracking. This dual-axis approach addresses significant limitations in existing narcotic cessation treatments by personalizing the intervention, reducing withdrawal severity, and improving patient compliance and overall engagement. This invention is particularly beneficial for clinical treatment programs targeting opioid dependence and similar substance use disorders.

Current therapeutic approaches for narcotic cessation, particularly with opioids, face substantial limitations in terms of effectiveness, patient compliance, relapse prevention, and long-term outcomes. Traditional treatments such as abrupt abstinence or basic medication-assisted therapies often lead to significant withdrawal symptoms, high dropout rates, psychological resistance, and relapse. These methods frequently lack structured protocols addressing both dosage reduction and the psychological aspect associated with varying ingestion methods. Additionally, there is an evident gap in the availability of integrated technological systems capable of actively guiding and monitoring patients through individualized treatment pathways.

The method proposed herein systematically addresses these limitations by establishing a clear, structured, dual-axis tapering framework. The framework simultaneously addresses the progressive reduction of narcotic dosage and transitions the patient sequentially through decreasingly reinforcing ingestion modalities. An ordered ingestion modality intensity hierarchy is established, ranking methods based on addiction reinforcement potential, including injection, smoking or vaping, insufflation, rectal administration, oral ingestion, and transdermal administration. The therapeutic method begins by identifying a patient's current ingestion modality on this hierarchy and guiding the patient toward transitioning to the next lower intensity modality. Following each transition, narcotic dosages are methodically reduced to allow gradual physiological and psychological adaptation. This iterative transition and dosage reduction continues until patients reach either complete cessation or a clinically defined minimal dosage within the lowest ingestion modality. The method includes patient pacing personalization, clinician oversight, and optional behavioral reinforcement mechanisms, such as randomized positive feedback, to improve engagement and reduce dropout rates.

Technologically, the invention addresses the technical challenge of guiding, monitoring, and dynamically adapting narcotic cessation treatments in real-time. Traditional treatments typically rely on clinician oversight and basic pharmacological interventions, lacking the capability for personalized, real-time adaptability. The present invention provides a sophisticated technological system to support and automate aspects of the described therapeutic method. This system includes digital components, such as a central computing platform or mobile application backend, patient-facing software applications, clinician dashboards, data analytics, adaptive algorithms, and optionally, smart drug dispensing hardware.

Patient software interfaces provide interactive features such as automated reminders, instructions for ingestion modality transitions, dosage tracking inputs, progress indicators, and motivational feedback. The software dynamically guides patients step-by-step through the structured ingestion ladder and dosage reduction protocol, based on inputs including current ingestion modality, adherence metrics, physiological indicators, and clinician-defined pacing. Clinician interfaces offer continuous monitoring and oversight, providing real-time patient adherence data, dosage adjustments, and patient-reported outcomes, allowing manual intervention as necessary. Integrated adaptive algorithms utilize machine learning to optimize individualized cessation regimens, dynamically adjusting modality transitions and dosage reductions based on patient feedback, adherence rates, and clinician inputs. An optional smart drug dispenser securely administers narcotic dosages according to prescribed regimens and automatically tracks adherence. Additionally, the technology incorporates behavioral reinforcement tools, including gamification and randomized reward mechanisms, to further enhance patient compliance.

Overall, this technological system operationalizes the narcotic cessation method in a structured yet adaptable manner, addressing physiological dependence, psychological reinforcement, patient adherence challenges, and clinical oversight requirements. The integrated technological solution significantly enhances treatment efficacy, patient experience, and outcomes in substance use disorder interventions, effectively bridging existing gaps in addiction treatment methodologies.

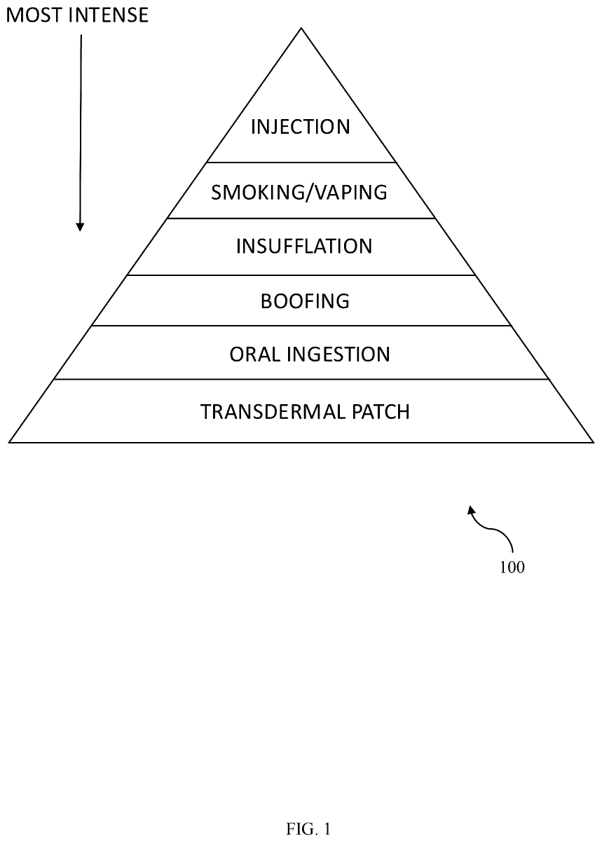

Referring now to the drawings in detail, FIG. 1 depicts an Ingestion Modality Hierarchy 100, a foundational element of the invention. This hierarchy organizes drug administration methods by their pharmacological and psychological reinforcement intensity. The top of the hierarchy lists Injection, which is considered the most intense due to its rapid onset, high bioavailability, and reinforcement potential. Below injection is Smoking/Vaping, a less intense yet still rapid delivery method. Following that are Insufflation (i.e., intranasal administration) and Boofing (i.e., rectal administration), which offer moderate onset and lower peak intensity. Next is Oral Ingestion, where the drug is consumed and metabolized more gradually. At the base of the hierarchy is Transdermal Patch, representing the least intense method due to its slow, steady drug release. This ladder visually encodes the therapeutic strategy of transitioning the patient downward, from the most to least reinforcing modality, as part of the treatment protocol.

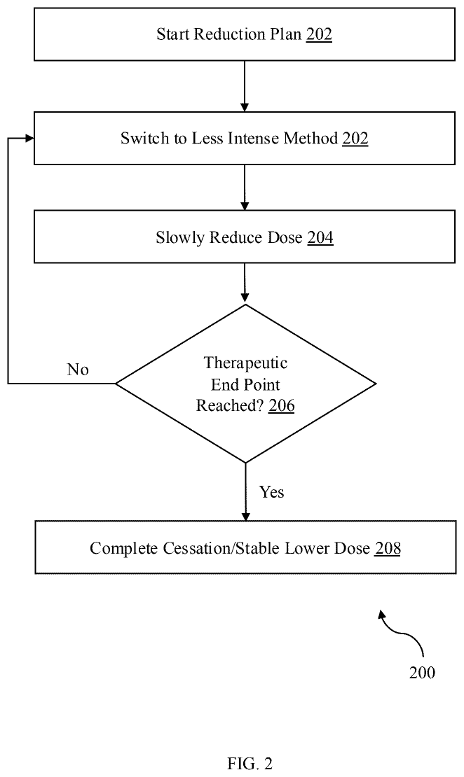

FIG. 2 illustrates the primary therapeutic method flow 200 used to guide patients through narcotic cessation. The process begins with a Start Reduction Plan 202, where a treatment program is initiated based on the patient's current behavior. The patient is prompted to Switch to a Less Intense Method 204, referencing the ingestion ladder of FIG. 1. Following this, the system instructs the patient to Slowly Reduce Dose 204, ensuring the individual remains physiologically stable. A Therapeutic Endpoint Check 206 evaluates whether the patient has reached an endpoint defined by either (a) complete cessation, or (b) a clinically acceptable minimal dosage within a low-intensity ingestion modality (such as a transdermal patch). If neither condition is met, the system loops back to suggest further reduction steps. Once the patient reaches either endpoint, the treatment concludes at the Therapeutic Endpoint Reached 208. Arrows indicate decision points (“Yes” or “No”) guiding progression based on treatment adherence and system recommendations.

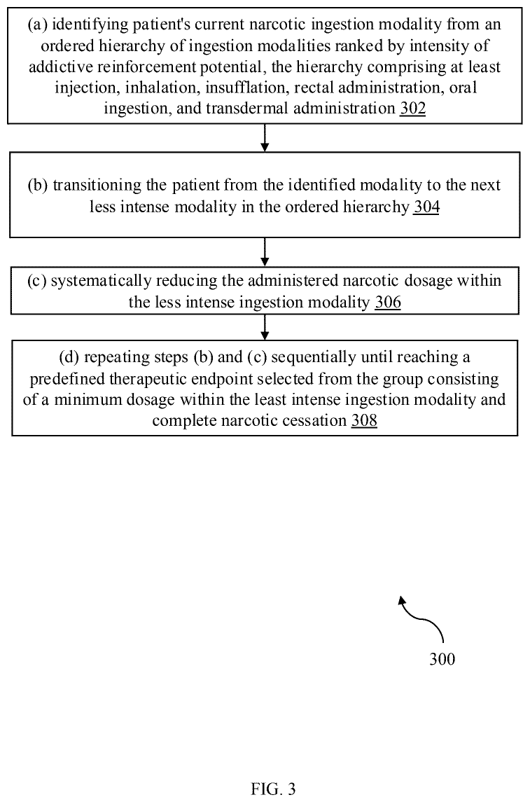

FIG. 3 presents a more granular view of the method steps 300 implemented by the system. The method begins with Step 302, which involves identifying a patient's current narcotic ingestion modality from the predefined hierarchy shown in FIG. 1. This step relies on clinician input, patient-reported data, or sensor input. Next, in Step 304, the patient is transitioned to the next less intense ingestion modality. This transition may be either automatic (via system logic) or clinician-guided. Step 306 requires a systematic reduction in the administered narcotic dosage within that less intense modality, ensuring physiological safety and treatment personalization. In Step 308, the system determines whether the cessation endpoint has been reached—either a very low dose within the lowest modality or complete abstinence. If not, steps 304 and 306 are repeated, progressively reducing the patient's dependence both behaviorally and chemically.

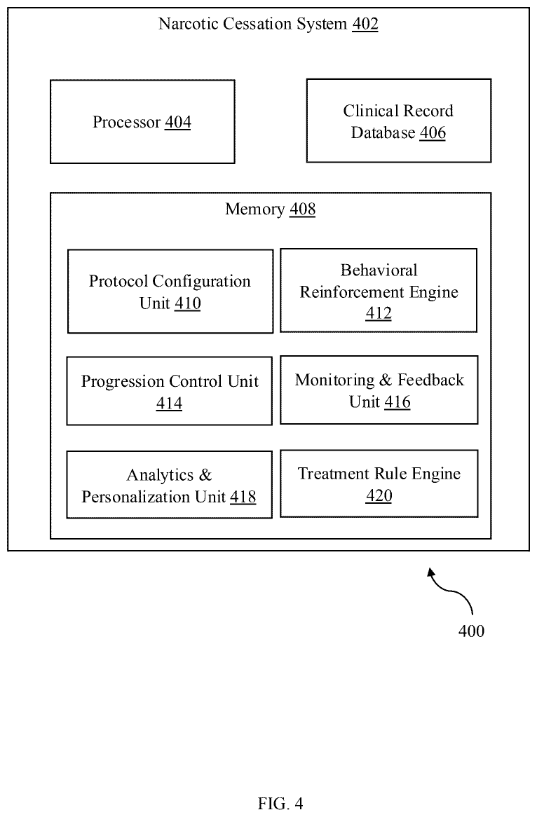

FIG. 4 shows the Narcotic Cessation System 400, a computing environment configured to execute the therapeutic method described in FIGS. 2 and 3. At the core is a Processor 404, which executes logic instructions and controls interactions between sub-components. The Memory 408 houses multiple system modules, including treatment logic, patient progress, and reinforcement engines. A Clinical Records Database 406 stores patient history, drug use patterns, ingestion method logs, clinician notes, and treatment outcomes, serving as both a record-keeping and decision-support resource.

Contained within the memory, the Protocol Configuration Unit 410 allows clinicians or system administrators to define ingestion hierarchies, tapering intervals, thresholds, and pacing parameters, enabling full customization. The Behavioral Reinforcement Engine 412 is configured to apply behavior-modification techniques, including randomized reinforcement schedules, gamification elements, and positive feedback cycles to increase adherence and reduce treatment dropout. The Progression Control Unit 414 manages the patient's advancement through ingestion modalities and dosage stages by evaluating thresholds, monitoring compliance, and suggesting appropriate transitions. The Monitoring and Feedback Unit 416 receives patient input, logs system interaction data, and may integrate biometric or adherence data to adjust pacing. Feedback may be delivered in real-time to the patient and clinician interface.

The Analytics and Personalization Unit 418 optionally applies statistical or machine-learning methods to personalize treatment parameters over time. For example, it may analyze the effectiveness of modality transitions, estimate relapse probability, or recommend individualized taper rates. Lastly, the Treatment Rule Engine 420 enforces pre-defined clinical policies, safety rules, and escalation logic. It may override automated transitions, delay progress, or alert clinicians if specific patterns indicate risk.

Together, these system components coordinate to deliver a personalized, adaptive, and structured narcotic cessation program. The configuration of software modules, logic control pathways, feedback integration, and clinical support distinguishes the invention from prior art and supports the dual-axis tapering approach (dose and modality) that underlies the method. Reference numerals throughout the figures correlate with specific components for consistency across this disclosure.

In a preferred embodiment, the system includes a computing device with a processor and memory configured to execute instructions for guiding a patient through the ingestion ladder and dosage tapering plan. The system retrieves and stores patient data in a clinical records database and may be accessed by a clinician through a secured dashboard interface. The memory includes several modules: a protocol configuration unit for defining the ingestion ladder and tapering plan, a progression control unit for tracking the patient's current modality and dosage, a behavioral reinforcement engine for delivering randomized encouragement or feedback, and a monitoring and feedback unit for collecting patient data and providing real-time updates. Optionally, the system includes an analytics and personalization module to adapt the plan to individual behaviors using heuristics or machine learning, and a treatment rule engine to enforce safety policies, block unsafe transitions, and trigger alerts.

In related embodiments, the system may be integrated with mobile applications, wearable devices, or biometric input sources. These tools enhance personalization by capturing real-time physiological and behavioral data. Clinicians may override automated decisions based on judgment or patient needs. The ingestion modality hierarchy may also be adjusted for specific drugs or populations, such as modifying the order or intensity weighting based on pharmacokinetics. Furthermore, the reinforcement strategies may include audio-visual prompts, milestone-based rewards, or randomized token economies to maintain patient engagement. These embodiments allow the invention to remain flexible, extensible, and adaptable to clinical requirements while preserving the core therapeutic protocol of progressive ingestion method reduction combined with dose tapering.

In related embodiments, the system may further include mechanisms for controlled behavioral variability, such as randomized feedback timing or entropy-driven dosing prompts. These enhancements may reduce psychological habituation by avoiding predictable reinforcement patterns, thereby improving long-term adherence and patient engagement. As used herein, ‘entropy’ refers to a measure of controlled unpredictability in timing or behavior, often generated using hardware-or cryptographic-based randomization sources to reduce anticipatory reinforcement patterns.

Deployment of the present invention may involve a standalone mobile application (for iOS or Android), a desktop software system, or a secure web-based clinician and patient dashboard. In certain embodiments, the system operates on a HIPAA-compliant cloud hosting environment that manages treatment logic, patient data logging, analytics, and remote clinical supervision. This centralized architecture enables seamless integration with third-party health platforms such as electronic medical records (EMRs), biometric data aggregators, or API-connected behavioral health systems. By incorporating individualized tapering schedules, ingestion modality transitions, clinician feedback loops, and optional reinforcement mechanisms—including randomization of dosage timing or feedback responses—the invention offers a significant improvement over conventional systems that rely on fixed, predictable schedules. It ensures both patient safety and adaptive personalization while intentionally disrupting psychological habituation linked to drug use.

In various embodiments, the features described herein are implemented using a combination of hardware, software, and firmware components operating within a unified or modular system architecture. On the software side, the invention comprises processor-executable instructions stored on non-transitory computer-readable media, including but not limited to flash memory, magnetic drives, solid-state storage, or secure cloud-based infrastructure. These instructions, when executed, perform critical operations such as ingestion method transition tracking, individualized tapering calculations, behavioral feedback scheduling, patient-clinician messaging, and secure event logging. The software may be delivered as a standalone application, a cloud-hosted backend accessible via web interface, or a distributed system managing mobile clients, clinical dashboards, and machine-learning personalization services.

The system may incorporate one or more processors, controllers, or microcontrollers configured to execute the aforementioned program logic. These processors manage the determination of appropriate ingestion modality transitions, generate tapered dose instructions based on clinical parameters, and deliver real-time user prompts and clinician alerts. In embodiments where behavioral reinforcement or entropy-enhanced variability is implemented (e.g., randomized timing of instructions), cryptographic or hardware-based entropy sources may be used to drive unpredictability within predefined therapeutic limits. Secure memory components, including secure enclaves or trusted execution environments, may be used to store sensitive data such as patient progress logs, tapering schedules, and authentication credentials, while ensuring compliance with data privacy regulations. In some embodiments, the processor may further interface with a randomization engine driven by a cryptographic entropy source, hardware-based random number generator, or software-based pseudorandom function. These may be used to introduce bounded variability in timing or feedback delivery within safe clinical constraints.

Memory subsystems include volatile memory such as DRAM or SRAM for executing runtime instructions, as well as non-volatile memory such as EEPROM, flash, or persistent cloud storage for long-term recordkeeping. These storage systems may hold user-specific ingestion histories, preference settings, tapering configurations, behavioral reinforcement thresholds, and real-time adherence metrics. In networked embodiments, data may be mirrored to cloud storage to support clinician review, multi-device access, automated redundancy, or regulatory compliance (e.g., HIPAA or GDPR).

Communication modules within the system may include secure interfaces for transmitting dosage and modality instructions to end-user devices via encrypted protocols over Wi-Fi, mobile broadband (4G/5G), or Bluetooth Low Energy. Depending on configuration, messages may be delivered via push notifications, secure in-app alerts, or token-protected communication channels. Integration with healthcare infrastructure may also be supported, including secure API-based access to EMRs, EHRs, or telemedicine systems. This allows bidirectional data flows between the invention's system and existing clinical workflows.

Scalable implementations of the invention may adopt a modular or service-oriented architecture, in which components such as the tapering configuration manager, behavioral engine, patient progression controller, clinician interface, and compliance logger operate independently. This modularity allows individual services to be updated, replaced, or scaled without interrupting the overall system. In cloud-enabled embodiments, the system supports over-the-air updates for configuration changes, treatment logic refinements, security patches, and regulatory adaptations, making it ideal for dynamic clinical environments.

The architecture of the present invention is engineered to provide a secure, configurable, and behaviorally intelligent environment for managing narcotic cessation. The system balances clinical safety with patient-centric flexibility, allowing it to disrupt harmful behavioral patterns, reinforce positive progress, and accommodate patient-specific recovery timelines. It may be deployed on a wide range of platforms, including smartphones, clinician workstations, and hybrid deployments utilizing cloud servers for scalability and fault tolerance.

In all embodiments, the programming logic and essential data structures may reside on non-transitory computer-readable media, or be transmitted via various digital or analog channels. These include radiofrequency, electromagnetic energy, optical signals, or internet-based protocols. Redundant and distributed data storage may be used to ensure resilience, enabling continuous system availability even during network or hardware failure. Optional security enhancements, including end-to-end encryption, blockchain transaction logging, or secure enclaves, may be deployed to ensure privacy, authenticity, and regulatory compliance.

Software deployment formats may include native mobile applications, responsive browser-based dashboards, and hybrid applications capable of running on embedded or wearable devices. In some implementations, patients may interact with the system via smartwatches, tablets, or in-home health terminals. Clinicians may monitor and adjust settings remotely. Connectivity may be maintained over Wi-Fi, 5G cellular networks, or local encrypted Bluetooth communications. These features ensure wide compatibility with current digital ecosystems and offer flexible modes of engagement tailored to user and clinical needs.

In certain embodiments, the system optionally incorporates behavioral variability mechanisms, including entropy-enhanced scheduling of instructions or feedback prompts. This controlled randomness may be bounded by predefined therapeutic parameters to preserve safety while reducing reinforcement loops that develop with fixed dosing schedules. Compared to conventional narcotic cessation strategies based on fixed-dose, fixed-timing regimens, this invention introduces a fundamentally new technical approach. Existing systems reinforce anticipatory psychological responses by delivering medication in predictable patterns, which can inadvertently trigger relapse or reduce compliance. In contrast, this embodiment introduces behavioral variability by optionally incorporating controlled randomness—such as entropy-driven feedback schedules or dosing reminders—bounded within clinically safe parameters. This disrupts reinforcement loops associated with timing anticipation, supporting better long-term adherence.

While the invention is primarily described in the context of narcotic or opioid cessation, its architecture and behavioral strategy may be adapted to other medical contexts where dosage tolerance, psychological conditioning, or compliance are challenges. This includes chronic pain treatment (e.g., to prevent tolerance to analgesics), psychiatric therapy involving habit-forming medications, or even lifestyle or wellness contexts such as caffeine or nicotine reduction. In clinical research, this system may support studies involving human behavior under randomized or adaptive dosing conditions. In certain embodiments, machine learning components may dynamically modify behavioral logic or taper pacing based on accumulated compliance and biomarker data.

Existing computerized medication systems focus on automating manual scheduling but fail to address the technical problem of psychological predictability. They do not incorporate ingestion modality transitions, nor do they adapt to behavioral reinforcement loops. As a result, these systems may perpetuate the very behaviors they are intended to treat. They also lack mechanisms for dynamically adjusting both route and dose based on feedback loops or clinician interventions.

In contrast, the invention described herein introduces a technically specialized behavioral tapering framework that integrates ingestion route hierarchy, algorithmic dose tapering, and optionally, randomization. This framework is enforced and tracked by a system architecture comprising patient-specific protocol engines, controlled feedback modules, and personalized control units. The randomness (when applied) is constrained within boundaries established by clinicians or treatment protocols, ensuring medical safety while reducing psychological habituation. The result is a behaviorally-informed, algorithmically-controlled narcotic cessation platform that responds dynamically to patient progress. Although the primary method of narcotic cessation does not require randomization, certain fallback implementations may incorporate entropy-controlled behavior modules to accommodate patients at high risk of relapse due to psychological conditioning. These modules, including randomized reward mechanisms or nondeterministic dose prompt timing, are designed to maintain novelty and engagement, particularly in long-duration tapering regimens.

The comprehensive architecture—including ingestion method management, personalized configuration, adaptive feedback, logging, clinician review, and optional AI-based taper adjustments—represents a significant technological improvement over existing treatment software. It exceeds mere automation by introducing clinically meaningful, personalized, and technically novel features that directly address the psychological and physiological complexities of addiction recovery. The invention therefore offers both therapeutic innovation and technological advancement, making it uniquely suited for scalable clinical and individual deployment.

It will be appreciated that while specific implementations of the invention have been described in detail, various modifications, enhancements, and adaptations may be made without departing from the spirit and scope of the invention. Accordingly, the described embodiments should not be construed as limiting but rather as illustrative of the broader capabilities of the invention.

It may be noted that the above-described examples of the present solution are for the purpose of illustration only. Although the solution has been described in conjunction with a specific embodiment thereof, numerous modifications may be possible without materially departing from the teachings and advantages of the subject matter described herein. Other substitutions, modifications, and changes may be made without departing from the spirit of the present solution. All the features disclosed in this specification (including any accompanying claims, abstract, and drawings), and all of the steps of any method or process so disclosed, may be combined in any combination, except combinations where at least some of such features or steps are mutually exclusive.

The terms “include,” “have,” and variations thereof, as used herein, have the same meaning as the term “comprise” or an appropriate variation thereof. Furthermore, the term “based on”, as used herein, means “based at least in part on.” Thus, a feature that is described as based on some stimulus can be based on the stimulus or a combination of stimuli including the stimulus.

The present description has been shown and described with reference to the foregoing examples. It is understood, however, that other forms, details, and examples can be made without departing from the spirit and scope of the present subject matter that is defined in the following claims.

What is claimed is:

1. A method for structured narcotic cessation comprising:
(a) identifying a patient's current narcotic ingestion modality from an ordered hierarchy of ingestion modalities ranked by intensity of addictive reinforcement potential, the hierarchy comprising at least injection, inhalation, insufflation, rectal administration, oral ingestion, and transdermal administration;
(b) transitioning the patient from the identified modality to the next less intense modality in the ordered hierarchy;
(c) systematically reducing the administered narcotic dosage within the less intense ingestion modality; and
(d) repeating steps (b) and (c) sequentially until reaching a predefined therapeutic endpoint selected from the group consisting of a minimum dosage within the least intense ingestion modality and complete narcotic cessation.

2. The method of claim 1, wherein the ordered hierarchy of ingestion modalities explicitly ranks ingestion methods based on at least one criterion selected from onset speed, bioavailability, addictive potential, or subjective intensity.

3. The method of claim 1, further comprising utilizing a randomized reinforcement mechanism to provide varied feedback or incentives to the patient to encourage continued adherence to modality transition and dosage reduction.

4. The method of claim 1, wherein systematic dosage reduction comprises predefined incremental dosage decrements based on clinical or pharmacokinetic criteria.

5. The method of claim 1, further comprising tracking patient progress through digital data logging accessible to the patient or healthcare provider.

6. The method of claim 1, wherein transitions between modalities are executed upon the detection of patient stability at the current modality, assessed through patient-reported outcomes, clinical assessment, or physiological biomarkers.

7. A narcotic cessation system comprising:
(a) a computing device comprising a processor and memory; and
(b) instructions stored on the memory, wherein the instructions, when executed by the processor, perform operations comprising:
(i) determining a patient's current narcotic ingestion modality from a predefined intensity-ranked ingestion modality hierarchy;
(ii) providing instructions or notifications prompting transition to a less intense ingestion modality from the ranked hierarchy;
(iii) calculating and recommending systematic dosage reductions within the less intense ingestion modality;
(iv) tracking patient adherence and progression through modalities and dosages; and
(v) identifying attainment of a cessation goal selected from either complete cessation or reaching a predefined minimal dosage within the lowest-intensity ingestion modality.

8. The narcotic cessation system of claim 7, further comprising integration with a smart drug dispenser configured to selectively dispense narcotics according to the calculated reduced dosage and ingestion modality.

9. The narcotic cessation system of claim 7, wherein the software instructions incorporate a machine-learning algorithm trained to dynamically adjust dosage reduction schedules based on patient feedback, adherence rates, or clinician input.

10. The narcotic cessation system of claim 7, further comprising a clinician interface configured to monitor patient progression, provide manual adjustments to modality transitions or dosage reductions, and enable communication between the clinician and patient.

11. The narcotic cessation system of claim 7, further comprising a gamification module configured to deliver rewards, incentives, or virtual achievements to patients upon reaching predefined progression milestones.

12. The narcotic cessation system of claim 7, wherein the software instructions are configured to provide patient-specific educational content relevant to each ingestion modality stage of cessation.

13. The narcotic cessation system of claim 7, wherein the predefined therapeutic endpoint comprises a transdermal narcotic patch modality at a clinically predetermined minimal dosage.

14. The narcotic cessation system of claim 7, wherein the instructions further include a randomization engine configured to introduce entropy-driven variability in dosage timing or feedback delivery within predefined safety parameters.
